(19) United States
(12) Patent Application Publication
(10) Pub. No.: US 2007/0106338 A1
(43) Pub. Date: May 10, 2007

(54) Direct and Indirect Control of Muscle for the Treatment of Pathologies

(75) Inventor: Joseph P. Errico, Green Brook, NJ (US)

Correspondence Address: KAPLAN GILMAN GIBSON & DERNIER L.L.P., 900 ROUTE 9 NORTH, WOODBRIDGE, NJ, 07095, US

(73) Assignee: ElectroCore, Inc., Summit, NJ (US)

(21) Appl. No.: 11/555,170

(22) Filed: Oct. 31, 2006

Related U.S. Application Data
(60) Provisional application No. 60/736,002, filed on Nov. 10, 2005.

(51) Int. Cl. A61N 1/36

(52) U.S. Cl. 607/42

(57) ABSTRACT

Methods for treating various health ailments that arise from dysfunction of smooth muscle associated with organ function caused by neurological malfunction wherein an appropriate signal fails to properly drive the smooth muscle wherein the treatment includes reproducing an appropriate signal and applying same either directly to the smooth muscle exhibiting the failure to respond properly, or by applying the signal to the nerves that innervate the muscles. The various treatments contemplated by this invention include, but are not limited to: acid reflux (lower esophageal sphincter), gallbladder pain, post-cholecystectomy pain, obesity, and high cholesterol (the sphincter of Oddi), pancreatic function and pancreatic pain associated with cancer (sphincter of Oddi), and asthma (the anterior and posterior pulmonary plexuses and the bronchial plexuses).

### CROSS REFERENCE TO RELATED APPLICATIONS

This application claims the benefit of U.S. Provisional Patent Application No: 60/736,002, filed Nov. 10, 2005, entitled DIRECT AND INDIRECT CONTROL OF MUSCLE FOR THE TREATMENT OF PATHOLOGIES, the entire disclosure of which is hereby incorporated by reference.

### FIELD OF THE INVENTION

The present invention relates to the field of electrical stimulation of bodily tissues for therapeutic purposes, and more specifically to treating ailments of the digestive, respiratory, and/or cardiovascular systems, and/or endocrine and renal functions, either by direct stimulation of the muscular tissues surrounding tubular tracts involved in the release or progression of solids, gasses and/or fluids therethrough, or indirect stimulation thereof by stimulation of the nerve fibers that innervate and regulate same.

### BACKGROUND OF THE INVENTION

The use of electrical stimulation has been well known in the art for nearly two thousand years. Roman physicians are reported to have used electric eels for treating headaches and pain associated with gout. In 1760, John Wesley used the primitive rudimentary electrical device, the Leyden Jar, was applied to therapeutic purposes hoping to shock patients suffering from paralysis, convulsions, seizures, headaches, angina, and sciatica.

It was not until Luigi Galvani, in 1791, that a disciplined study of the effects of electricity on muscles and nerves was done in a scientifically rigorous manner. In 1793, Alessandro Volta furthered this work when he reported that muscle contraction could be forced to occur when an electrified metal was placed in the vicinity of a motor nerve and the muscle innervated by that nerve.

One of the most successful modern applications of this basic understanding of the relationship between muscle and nerves is the cardiac pacemaker. Although its roots extend back into the 1800's, it wasn't until 1950 that the first practical, albeit external and bulky pacemaker was developed. Dr. Rune Elqvist developed the first truly functional, wearable pacemaker in 1957. Shortly thereafter, in 1960, the first fully implanted pacemaker was developed. Around this time, it was also found that the electrical leads could be connected to the heart through veins, which eliminated the need to open the chest cavity and attach the lead to the heart wall. In 1975 the introduction of the lithium-iodide battery prolonged the battery life of a pacemaker from a few months to more than a decade. The modern pacemaker can treat a variety of different signaling pathologies in the cardiac muscle, and can serve as a defibrillator as well (see U.S. Pat. No. 6,738,667 to Deno, et al., the disclosure of which is incorporated herein by reference).

The application of this electrical stimulation to the nervous system for other medical applications, of course, includes electroshock therapy for mental illness, such as for schizophrenia and depression. Early brute force attempts to apply voltage across the skull have, thankfully, evolved to the point where leads are being implanted into very specifically mapped regions of the brain, so that precise amounts of electricity can be applied far more effectively, and with far fewer complications (see U.S. Pat. No. 6,871,098 to Nuttin, et al., the disclosure of which is incorporated herein by reference).

The applications for deep brain stimulation go beyond simply mental illness of a behavioral nature, but also extend to degenerative motor dysfunctions associated with brain-based pathologies, such as Parkinsons disease and essential tremor (see, for example, Meadows, et al. U.S. Pat. No. 6,920,359, the teachings and specification of which are incorporated herein by reference). Certain facial and body pain can be treated by applying electrical stimulation to the surface of the brain as well, for example, see U.S. Pat. No. 6,735,475 to Whitehurst, et al., the disclosure of which is incorporated herein by reference.

Another application of electrical stimulation of nerves has been the treatment of radiating pain in the lower extremities by means of stimulation of the sacral nerve roots at the bottom of the spinal cord (see U.S. Pat. No. 6,871,099 to Whitehurst, et al., the disclosure of which is incorporated herein by reference).

Just as the stimulation of the brain can be used to treat pain and motor function pathologies in the body, nerve stimulation in the periphery can be used to affect the behavior of patients. For example, treatments for depression and overeating have been utilized with varying degrees of reported success within the past decade.

Organ Function: Many bodily functions necessary for survival are carried out or assisted by the organs of the thoracic and abdominal cavities. These organs are often tightly associated with musculature that may control the flow of secretions, food matter, oxygen, and waste matter along the paths they are to travel.

For example, the esophageal sphincter is the muscular valve that opens to permit food to enter the stomach, and tightens to prevent the acidic stomach contents to rise up in the esophagus when the body is prone, or otherwise when pressure within the stomach rises. Research has suggested that nerve fibers from the greater splanchnic nerves of the sympathetic nerve chain as well as nerve fibers from the cervical and vagus nerve branches innervate the plexuses along the esophagus to control the muscle activity in the sphincter. A failure of this sphincter to function properly can lead to acid reflux and heartburn, with or without regurgitation of gastric contents. Complications of this can include esophagitis, esophageal stricture, and esophageal ulcer, which can lead to odynophagia and even hemorrhage, which can be massive. Sharma disclosed, in U.S. Pat. No. 6,901,295, a method and apparatus for electrically stimulating the lower esophageal sphincter to control its function. The disclosure of the U.S. Pat. No. 6,901,295 is incorporated herein by reference.

The pyloric sphincter (also known as the pyloric valve), at the distal end of the stomach is also a series of muscles that open and shut the bottom of the stomach to control the flow of food contents from the stomach on their course into the duodenum. The muscles of this sphincter appear to be controlled by nerves of the greater and lesser splanchnic nerve fibers which emanate from the 5^{th} to the 12^{th} thoracic regions of the sympathetic nerve chain, as well as afferent fibers of the right branch of the vagus nerve. Failure of the pyloric sphincter to function properly can cause a variety of pathologies. One common malady for infants is pyloric stenosis in which the infant's muscles in the pyloric sphincter become enlarged such that food cannot pass through the stomach and into the duodenum. The treatment of this has traditionally been to perform an invasive procedure referred to as a pyloromyotomy. Alternatively, a failure of the pyloric sphincter to remain closed during digestion can lead to blockages within the intestines as food matter advances into the intestines without having been fully digested.

The flow of bile from the liver into the digestive tract, either from the lower portions of the liver that first passes into the gallbladder and then into the common bile duct, or from the superior portions of the liver and directly into the common bile duct, is ultimately regulated by the sphincter of Oddi. The failure of this sphincter to function properly and permit the flow of bile as needed (stenosis or other spasmodic dysfunction) may cause a build up of bile pressure in the branches of the bile duct, causing a distension of the gallbladder. Crystallization of the cholesterol present in the bile can lead to stones, and the ultimate removal of the gallbladder.

With the advent of laproscopic surgical techniques, cholecystectomies (removals of the gallbladder) are being performed at the rate of over five hundred thousand per year in the United States alone. While this procedure may alleviate the acute pathology of stones in the gallbladder, it may not resolve the sphincter problem, and may in fact exacerbate the problem as the bile that is trapped behind the dysfunctional sphincter can build up under pressure that is not regulated by the presence of the gallbladder (expansion of which may serve to relieve hypertension in the bile duct), causing excruciating pain, often referred to as post-cholecystectomy syndrome or PCS. PCS associated with sphincter of Oddi dysfunction has been estimated to be a problem for upwards of 10-15% of all patients who have undergone cholecystectomies. It is unclear at this time what percentage of patients presently undergoing gallbladder removals would be better treated for sphincter of Oddi dysfunction directly.

Typical treatments for PCS pain include the placement of a stent in the sphincter to prevent closure (see U.S. Pat. No. 5,876,450 to Johlin, U.S. Pat. No. 5,282,824 and 5,507,771 to Gianturco, U.S. Pat. No. 5,486,191 and 5,776,160 to Pasricha, et al., the disclosures of which are incorporated herein by reference), botulism toxin injections to paralyze the muscle of the sphincter (see U.S. Pat. Nos. 5,437,291 and 5,674,205 to Pasricha, et al., the disclosures of which are incorporated herein by reference), and surgically cutting the muscles (a sphincterotomy). Clinical research in Turkey, reported by Guler, et al. (see Turkish Journal of Medical Sciences, Vol. 29 (1999) p 303-307, the teachings of which are incorporated herein by reference) has suggested that physical destruction (i.e., cutting) of the hepatic plexus can have an equivalent effect as sphincterotomy. This would entail cutting the distal fibers of the splanchnic nerves and fibers of the left vagus nerve, as they are the neurons that form the hepatic plexus.

Forced relaxation and or destruction of the muscles that form the sphincter of Oddi by any of these means, however, has been associated with dramatic hunger pains that arise after any prolonged period of fasting. Patients who have undergone sphincteromies of these muscles, for example, have complained of such profound hunger that it disrupts their sleep at night, virtually forcing them to eat additional meals and leaving them gravely disadvantaged in attempts to control their weight.

The free flow of bile into the gut has another potentially significant consequence related to blood cholesterol levels. The liver is a primary producer of cholesterol for a variety of uses, including the synthesis of hormones and cell membranes. This cholesterol enters the bloodstream through the bile flow into the gut, and its direct absorption into the bloodstream through the intestinal wall. Free flow of bile in the gut, therefore can theoretically cause a rise in cholesterol levels. This is true, not only because of the physical presence of bile in the gut, but also because of the inherent inhibitory effect cholesterol has on the continued production of more cholesterol.

More specifically, the hepatic cells of the liver produce cholesterol through a biosynthesis process that includes an enzyme known as HOA-C. This enzyme serves as a regulator for the process inasmuch as cholesterol can competitively bind to the enzyme, shutting the enzyme off. The presence of cholesterol in sufficient concentrations, therefore, causes the enzyme to stop the synthesis. Most of the major anti-cholesterol drugs, including Lipitor, Zocor, Pravachol, Mevacor, and Vytorin leverage this fact by incorporating a moiety in their molecular structure that mimics the portion of cholesterol that competitively binds to HOA-C, thus inhibiting cholesterol synthesis. The free flow of bile out of the liver, along with the cholesterol in it, without any inhibition may eliminate an important regulatory effect preventing the overproduction of cholesterol.

It should also be recognized that, while hypertension in the bile duct can cause excruciating pain, hypotension in the bile duct because of a failure of the sphincter of Oddi to maintain proper function may result in indigenously high cholesterol levels, the same way that surgically opening the sphincter can. Similarly, it is also possible that obesity in some individuals may be attributed to a low tonicity in the sphincter of Oddi because the constant presence of bile in the gut causes persistent hunger sensations.

The pancreas also produces secretions that are critical to proper digestion. These include some of the most powerful protolytic enzymes, including amylase, trisinogen, chymotrisinogen, and pancreatic lipase. The sphincter of Oddi also regulates the flow of these secretions into the digestive tract. Dysfunction of this sphincter can, therefore, cause a host of pathologies associated with the pancreas. Botulism toxin has been used to force the opening of this sphincter in this application as well (see U.S. Pat. Nos. 6,143,306 and 6,261,572 to Donovan the disclosures of which are incorporated herein by reference).

It has been suggested by some researchers that the intractable pain associated with terminal pancreatic cancer is the result of hypertension within the pancreas, and can therefore be relieved with a sphincterotomy or the severing of the nerves that control the sphincter of Oddi, both having the effect of permitting the free flow of secretions from the pancreas into the gut.

There are several other sphincters associated with the digestive tract, all of which are controlled by muscles that are innervated and directed by nerve plexuses that associate with the fibers of the sympathetic nerve chain (which interfaces with the spinal cord nerve roots), and the major peripheral nerves throughout the thoracic, abdominal, and even the pelvic cavities.

In addition to the digestive system, the smooth muscles that line the bronchial passages are controlled by a similar confluence of vagus and sympathetic nerve fiber plexuses. Spasms of the bronchi during asthma attacks can often be directly related to pathological signaling within these plexuses.

Similarly, renal and bladder function is critically dependent upon proper functioning of the sphincters associated with these organs. More specifically, the renal, hypogastric, superior and inferior mesenteric plexuses control a series of sphincters, including the internal urethral sphincter.

### SUMMARY OF THE INVENTION

In a first embodiment, the present invention contemplates an electrical stimulation device that drives an excitatory and/or non-excitatory signal to the muscle fibers surrounding an anatomical passageway through which materials critical for life, i.e., food matter, digestive fluids, waste products, blood, and/or air may travel. In a second embodiment, the present invention contemplates an electrical stimulations device that drives an excitatory and/or non-excitatory signal to the nerve plexus and/or surrounding nerve tissues controlling muscle fibers surrounding an anatomical passageway through which materials critical for life, i.e., food matter, digestive fluids, waste products, blood, and/or air may travel.

In preferred embodiments that are related to the digestive system, the stimulation signals are applied in a manner that relaxes and/or flexes sphincter muscles to permit or prevent passage of material through the duct or passageway around which the sphincter is associated. For example, the lower esophageal sphincter may be caused to tighten and close in patients for whom reflux of stomach contents into the esophagus is determined to be occurring. Alternatively, stimulation to relax the lower esophageal sphincter may be applied when stricture, or pathological closure of the sphincter, is identified. It shall be understood that the activation of such signals may be directed manually by the patient, or automatically through a feedback mechanism that recognizes and responds to a state of the stomach, the signals in the nerves that ordinarily direct the operation of the lower esophageal sphincter, and/or the esophagus itself. For example, the pH of the esophagus may be monitored, and when it is found to rise above a threshold level, the tightening of the esophagus could be triggered to prevent reflux of acidic stomach contents into the esophagus.

In distinct preferred embodiments that are related to the respiratory system, the stimulation signals are applied in a manner that relaxes the bronchi and/or the smooth muscle lining the bronchial passages to relieve the spasms that occurs during asthma attacks. As above, the stimulation signals may be applied by positioning leads on the muscle, bronchial tissue, or the nerves that control bronchial activity such as the anterior and posterior bronchial branches of the right and left branches of the vagus nerve, which join with fibers from the sympathetic nerve chain to form the anterior and posterior pulmonary plexuses. It shall also be understood that leadless stimulation, as shown in the art may also be utilized for applying stimulation to these tissues and/or plexuses, as well as the tissues of the digestive tract discussed above. In this respiratory application, the stimulation signal may be provided solely to arrest the spasms, or it may be applied to completely relax the tissue.

The mechanisms by which the appropriate stimulation is applied to the target tissue can include positioning the distal ends of an electrical lead or leads in the vicinity of the muscle and/or the nervous tissue controlling the sphincter, which leads are coupled to an implantable or external electrical signal generating device. The electric field generated at the distal tip of the lead creates a field of effect that permeates the target tissue (muscle or nerve fibers) and cause the excitation or relaxation of the muscle of the sphincter.

In yet another preferred embodiment, the target tissues are the sphincter muscles that control urine flow into the bladder through the ureters, and from the bladder into the urethra during urination. Electrical signals that quiet the spasms of these ducts can reduce the number of urination occurrences, and tightening these muscles can reduce incidents of incontinence.

In a distinct preferred embodiment, the sphincter of Oddi may be stimulated by direct application of electrical stimulation to the smooth muscles of the sphincter, or by modulation of the signals applied to the sphincter through the hepatic plexus. It shall be understood that the control of this sphincter includes three separate passages through which fluids may pass.

The first is the duct extending from the pancreas to the common bile duct, through which pancreatic enzymes and digestive fluids pass. Failure of this portion of the sphincter to properly function can result in (i) hypertension of these enzymes in the pancreas, which can cause significant pain, or (ii) reflux of bile and other digestive material into the pancreas, which causes the activation of the pancreatic enzymes and the resulting autodigestion of the pancreas which is exceptionally painful and damaging to the organ. Spasms in this portion of the muscle can result in either of the above effects, and is considered pathological. Stimulation to reduce spasms, or to relax and/or tighten this portion of the smooth muscle is critical to preventing pancreatitis and/or relieving the intractable pain associated with pancreatic cancer.

The second is the duct through which bile travels from the liver and gallbladder into the common bile duct. Hypertension in this duct can result in exceptionally painful sensations. This hypertension is alleviated when the gallbladder is present and functioning, as the bladder expands. The presence of large quantities of bile that is not able to flow can cause the cholesterol within the fluid to crystallize, ultimately forming stones that can result in blockage and/or pain. Control of this portion of the sphincter complex may be managed by direct application of stimulation signals to the muscles that form it, or by applying stimulation to the nerves that innervate the muscle, i.e., nerves passing into, and out from the hepatic plexus.

The third valve of the sphincter of Oddi is the one that releases the bile and pancreatic fluids into the digestive tract. Failure of this valve to function correctly by excessive laxity can cause a free flow of bile into the digestive tract at all times, or a reflux of the non-sterile food matter into the bile duct, causing potential infection. Free flow of bile into the digestive tract can have a number of deleterious effects, including excessive and persistent hunger pains, elevated cholesterol levels (associated with a constant flow of cholesterol from the liver into the intestines and into the bloodstream), and damage to the lining of the digestive tract.

Failure of this third valve by excessive tightening has the same effect as the excessive tightening of either of the pancreatic or bile duct valves discussed above, i.e., reflux of bile into the pancreas, hypertension in the pancreas or bile duct, the formation of gallbladder stones, as well as reflux of pancreatic fluids into the upper bile duct, which can result in significant damage to both organs and all of the structures associated with them.

It shall be understood that the complex of three separate muscle sphincters are implicated with these functions, and applying a laxity stimulation across hepatic plexus may drive the entire complex to laxity, resulting in reflux. Similarly, driving tightening through all three valve muscles may result in hypertension in one or both organs. Spasms, however, can more often be reduced by a simple signal that does not reduce the ability of the non-pathological indigenous signals from driving appropriate function. Therefore, it shall be understood, that with respect to the sphincter of Oddi, and the complex of valves associated with it, it may be preferred that there be a multiple lead stimulation unit used, with one lead positioned in contact with each cluster of muscles controlling the valves (or one lead positioned on the muscle of the two valves that are proximal to the opening into the digestive tract, i.e., the first and second valves set forth above, as they are formed by a single set of muscles that form a figure-8 structure about both merging branches of the duct).

The application of electrical stimulation, either to the nerve plexus or directly into the muscle to relax spasm, reduce excessive tension in the muscle, or induce a tightening of the muscle is more completely described in the following detailed description of the invention, with reference to the drawings provided herewith, and in claims appended hereto.

### DESCRIPTION OF THE DRAWINGS

For the purposes of illustration, there are forms shown in the drawings that are presently preferred, it being understood, however, that the invention is not limited to the precise arrangements and instrumentalities shown.

FIG. 1 is a schematic diagram of the human autonomic nervous system, illustrating sympathetic fibers, spinal nerve root fibers, and cranial nerves;

FIGS. 2-4 are various views of the anatomy of a human liver; and

FIG. 5 is a graphical illustration of an electrical signal profile that may be used to treat disorders through neuromuscular modulation in accordance with one or more embodiments of the present invention.

### DETAILED DESCRIPTION OF THE PREFERRED EMBODIMENTS

It shall be understood that the embodiments disclosed herein are representative of preferred aspects of the invention and are so provided as examples of the invention. The scope of the invention, however, shall not be limited to the disclosures provided herein, but solely by the claims appended hereto.

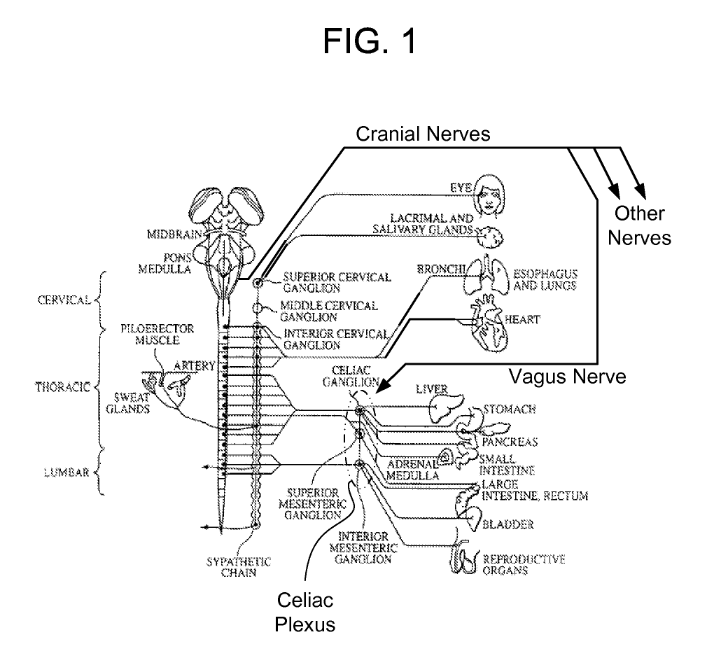

With reference to the drawings wherein like numerals indicate like elements there is shown in FIG. 1 a schematic diagram of the human autonomic nervous system, including sympathetic fibers, parasympathetic fibers, and cerebral nerves.

The sympathetic nerve fibers, along with many of the spinal cord's nerve root fibers, and the cranial nerves that innervate tissue in the thoracic and abdominal cavities are sometimes referred to as the autonomic, or vegetative, nervous system. The sympathetic, spinal, and cranial nerves all have couplings to the central nervous system, generally in the primitive regions of the brain, however, these components have direct effects over many regions of the brain, including the frontal cortex, thalamus, hypothalamus, hippocampus, and cerebellum. The central components of the spinal cord and the sympathetic nerve chain extend into the periphery of the autonomic nervous system from their cranial base to the coccyx, essentially passing down the entire spinal column, including the cervical, thoracic and lumbar regions. The sympathetic chain extends on the anterior of the column, while the spinal cord components pass through the spinal canal. The cranial nerves, the one most innervating of the rest of the body being the vagus nerve, passes through the dura mater into the neck, and then along the carotid and into the thoracic and abdominal cavities, generally following structures like the esophagus, the aorta, and the stomach wall.

In one embodiment, the invention comprises a method of treating asthmatic spasms. It comprises applying an electrical stimulation signal to at least one smooth muscle disposed in the vicinity of bronchial tissues whereby relaxation of said at least one smooth muscle is affected such that proper functioning of the bronchial tissues is permitted. Alternatively, this method of treating asthmatic spasms of bronchial tissue may comprise applying an electrical stimulation signal to at least one nerve fiber such that relaxation of at least one smooth muscle disposed in the vicinity of the patient's bronchial tissues is affected and proper functioning of the bronchial tissue is permitted.

This method of applying the electrical stimulation signal to at least one nerve fiber may further be refined such that the at least one nerve fiber comprises at least one nerve emanating from a patient's sympathetic nerve chain. Similarly, the at least one nerve may comprise at least one nerve fiber emanating from the patient's tenth cranial nerve (the vagus nerve), and in particular, at least one of the anterior bronchial branches thereof, or alternatively at least one of the posterior bronchial branches thereof. Preferably the stimulation is provided to at least one of the anterior pulmonary or posterior pulmonary plexuses aligned along the exterior of the lung. As necessary, the stimulation may be directed to nerves innervating the bronchial tree and lung tissue itself.

Additional details of the human autonomic nervous system of FIG. 1 are provided below, which will illustrate how the electrical stimulation of nerves and muscles in accordance with various embodiments of the present invention may be carried out. Because the autonomic nervous system has both afferent and efferent components, modulation of its fibers can affect both the end organs (efferent) as well as the brain structure to which the afferents fibers are ultimately coupled within the brain.

Although sympathetic and cranial fibers (axons) transmit impulses producing a wide variety of differing effects, their component neurons are morphologically similar. They are smallish, ovoid, multipolar cells with myelinated axons and a variable number of dendrites. All the fibers form synapses in peripheral ganglia, and the unmyelinated axons of the ganglionic neurons convey impulses to the viscera, vessels and other structures innervated. Because of this arrangement, the axons of the autonomic nerve cells in the nuclei of the cranial nerves, in the thoracolumbar lateral comual cells, and in the gray matter of the sacral spinal segments are termed preganglionic sympathetic nerve fibers, while those of the ganglion cells are termed postganglionic sympathetic nerve fibers. These postganglionic sympathetic nerve fibers converge, in small nodes of nerve cells, called ganglia that lie alongside the vertebral bodies in the neck, chest, and abdomen. The effects of the ganglia as part of the autonomic system are extensive. Their effects range from the control of insulin production, cholesterol production, bile production, satiety, other digestive functions, blood pressure, vascular tone, heart rate, sweat, body heat, blood glucose levels, and sexual arousal.

The parasympathetic group lies predominately in the cranial and cervical region, while the sympathetic group lies predominantly in the lower cervical, and thoracolumbar and sacral regions. The sympathetic peripheral nervous system is comprised of the sympathetic ganglia that are ovoid/bulb like structures (bulbs) and the paravertebral sympathetic chain (cord that connects the bulbs). The sympathetic ganglia include the central ganglia and the collateral ganglia.

The central ganglia are located in the cervical portion, the thoracic portion, the lumbar portion, and the sacral portion. The cervical portion of the sympathetic system includes the superior cervical ganglion, the middle cervical ganglion, and the interior cervical ganglion.

The thoracic portion of the sympathetic system includes twelve ganglia, five upper ganglia and seven lower ganglia. The seven lower ganglia distribute filaments to the aorta, and unite to form the greater, the lesser, and the lowest splanchnic nerves. The greater splanchnic nerve (splanchnicus major) is formed by branches from the fifth to the ninth or tenth thoracic ganglia, but the fibers in the higher roots may be traced upward in the sympathetic trunk as far as the first or second thoracic ganglion. The greater splanchnic nerve descends on the bodies of the vertebrae, perforates the crus of the diaphragm, and ends in the celiac ganglion of the celiac plexus. The lesser splanchnic nerve (splanchnicus minor) is formed by filaments from the ninth and tenth, and sometimes the eleventh thoracic ganglia, and from the cord between them. The lesser splanchnic nerve pierces the diaphragm with the preceding nerve, and joins the aorticorenal ganglion. The lowest splanchnic nerve (splanchnicus imus) arises from the last thoracic ganglion, and, piercing the diaphragm, ends in the renal plexus.

The lumbar portion of the sympathetic system usually includes four lumbar ganglia, connected together by interganglionic cords. The lumbar portion is continuous above, with the thoracic portion beneath the medial lumbocostal arch, and below with the pelvic portion behind the common iliac artery. Gray rami communicantes pass from all the ganglia to the lumbar spinal nerves. The first and second, and sometimes the third, lumbar nerves send white rami communicantes to the corresponding ganglia.

The sacral portion of the sympathetic system is situated in front of the sacrum, medial to the anterior sacral foramina. The sacral portion includes four or five small sacral ganglia, connected together by interganglionic cords, and continuous above with the abdominal portion. Below, the two pelvic sympathetic trunks converge, and end on the front of the coccyx in a small ganglion.

The collateral ganglia include the three great gangliated plexuses, called, the cardiac, the celiac (solar or epigastric), and the hypogastric plexuses. The great plexuses are respectively situated in front of the vertebral column in the thoracic, abdominal, and pelvic regions. They consist of collections of nerves and ganglia; the nerves being derived from the sympathetic trunks and from the cerebrospinal nerves. They distribute branches to the viscera.

Although all of the great plexuses (and their sub-parts) are of interest in accordance with various embodiments of the present invention, by way of example, the celiac plexus is shown in FIG. 1 in more detail. The celiac plexus is the largest of the three great sympathetic plexuses and is located at the upper part of the first lumbar vertebra. The celiac plexus is composed of the celiac ganglia and a network of nerve fibers uniting them together. The celiac plexus and the ganglia receive the greater and lesser splanchnic nerves of both sides and some filaments from the right vagus nerve. The celiac plexus gives off numerous secondary plexuses along the neighboring arteries. The upper part of each celiac ganglion is joined by the greater splanchnic nerve, while the lower part, which is segmented off and named the aorticorenal ganglion, receives the lesser splanchnic nerve and gives off the greater part of the renal plexus.

The secondary plexuses associated with the celiac plexus consist of the phrenic, hepatic, lineal, superior gastric, suprarenal, renal, spermatic, superior mesenteric, abdominal aortic, and inferior mesenteric. The phrenic plexus emanates from the upper part of the celiac ganglion and accompanies the inferior phrenic artery to the diaphragm, with some filaments passing to the suprarenal gland and branches going to the inferior vena cava, and the suprarenal and hepatic plexuses. The hepatic plexus emanates from the celiac plexus and receives filaments from the left vagus and right phrenic nerves. The hepatic plexus accompanies the hepatic artery and ramifies upon its branches those of the portal vein in the substance of the liver. Branches from hepatic plexus accompany the hepatic artery, the gastroduodenal artery, and the right gastroepiploic artery along the greater curvature of the stomach.

The lienal plexus is formed from the celiac plexus, the left celiac ganglion, and from the right vagus nerve. The lienal plexus accompanies the lienal artery to the spleen, giving off subsidiary plexuses along the various branches of the artery. The superior gastric plexus accompanies the left gastric artery along the lesser curvature of the stomach, and joins with branches from the left vagus nerve. The suprarenal plexus is formed from the celiac plexus, from the celiac ganglion, and from the phrenic and greater splanchnic nerves. The suprarenal plexus supplies the suprarenal gland. The renal plexus is formed from the celiac plexus, the aorticorenal ganglion, and the aortic plexus, and is joined by the smallest splanchnic nerve. The nerves from the suprarenal plexus accompany the branches of the renal artery into the kidney, the spermatic plexus, and the inferior vena cava.

The spermatic plexus is formed from the renal plexus and aortic plexus. The spermatic plexus accompanies the internal spermatic artery to the testis (in the male) and the ovarian plexus, the ovary, and the uterus (in the female). The superior mesenteric plexus is formed from the lower part of the celiac plexus and receives branches from the right vagus nerve.

The superior mesenteric plexus surrounds the superior mesenteric artery and accompanies it into the mesentery, the pancreas, the small intestine, and the great intestine. The abdominal aortic plexus is formed from the celiac plexus and ganglia, and the lumbar ganglia. The abdominal aortic plexus is situated upon the sides and front of the aorta, between the origins of the superior and inferior mesenteric arteries, and distributes filaments to the inferior vena cava. The inferior mesenteric plexus is formed from the aortic plexus. The inferior mesenteric plexus surrounds the inferior mesenteric artery, the descending and sigmoid parts of the colon and the rectum.

While the sympathetic and parasympathetic nervous system extends between the brain and the great plexuses, the cranial nerves extend between the brain and the great plexuses along other paths. For example, the sympathetic and parasympathetic nerves extend between the brain the celiac plexus, while the vagus nerve extends between the brain the celiac plexus along a second portion of the same circuit.

There are twelve pairs of cranial nerves, namely: the olfactory, optic, oculomotor, trochlear, trigeminal, abducent, facial, acoustic, glossopharyngeal, vagus (the tenth cranial nerve), accessory, and hypoglossal. The nuclei of origin of the motor nerves and the nuclei of termination of the sensory nerves are brought into relationship with the cerebral cortex.

Although all of the cranial nerves are of interest in accordance with various embodiments of the present invention, by way of example, the vagus nerve is shown in FIG. in more detail. The vagus nerve is composed of motor and sensory fibers and is of considerable interest in connection with various embodiments of the present invention because it has a relatively extensive distribution than the other cranial nerves and passes through the neck and thorax to the abdomen. The vagus nerves leave the cranium and are contained in the same sheath of dura mater with the accessory nerve. The vagus nerve passes down the neck within the carotid sheath to the root of the neck. On the right side, the nerve descends by the trachea to the back of the root of the lung, where it spreads out in the posterior pulmonary plexus. From the posterior pulmonary plexus, two cords descend on the esophagus and divide to form the esophageal plexus. The branches combine into a single cord, which runs along the back of the esophagus, enters the abdomen, and is distributed to the posteroinferior surface of the stomach, joining the left side of the celiac plexus, and sending filaments to the lienal plexus.

On the left side, the vagus nerve enters the thorax, crosses the left side of the arch of the aorta, and descends behind the root of the left lung, forming the posterior pulmonary plexus. From posterior pulmonary plexus, the vagus nerve extends along the esophagus, to the esophageal plexus, and then to the stomach. The vagus nerve branches over the anterosuperior surface of the stomach, the fundus, and the lesser curvature of the stomach.

The branches of distribution of the vagus nerve are as follows: the auricular, the superior laryngeal, the recurrent, the superior cardiac, the inferior cardiac, the anterior bronchial, the posterior bronchial, the esophageal, the celiac, and the hepatic. Although all of the branches of the vagus nerve are of interest in accordance with various embodiments of the invention, the gastric branches and the celiac branches are believed to be of notable interest. The gastric branches are distributed to the stomach, where the right vagus nerve forms the posterior gastric plexus on the postero-inferior surface of the stomach and the left vagus nerve forms the anterior gastric plexus on the antero-superior surface of the stomach. The celiac branches are mainly derived from the right vagus nerve, which enter the celiac plexus and supply branches to the pancreas, spleen, kidneys, suprarenal bodies, and intestine.

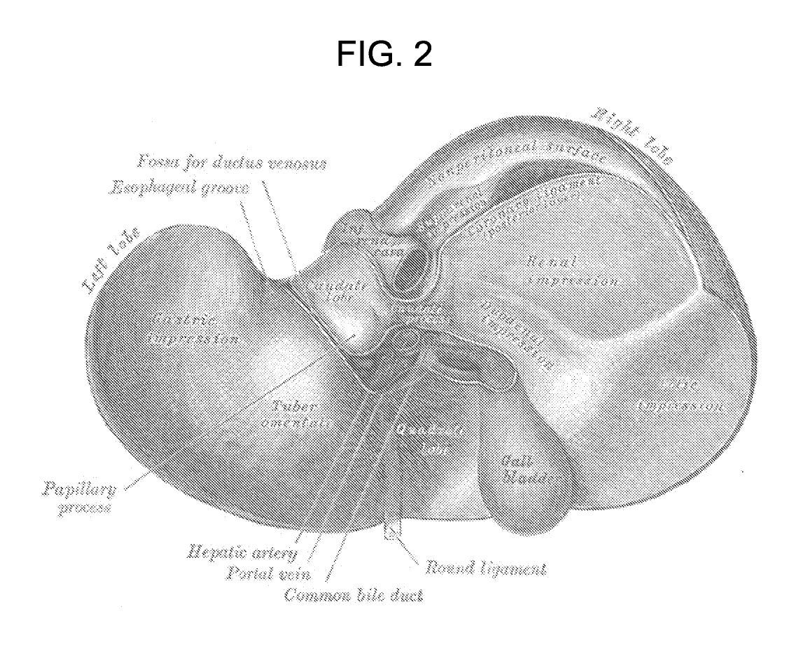

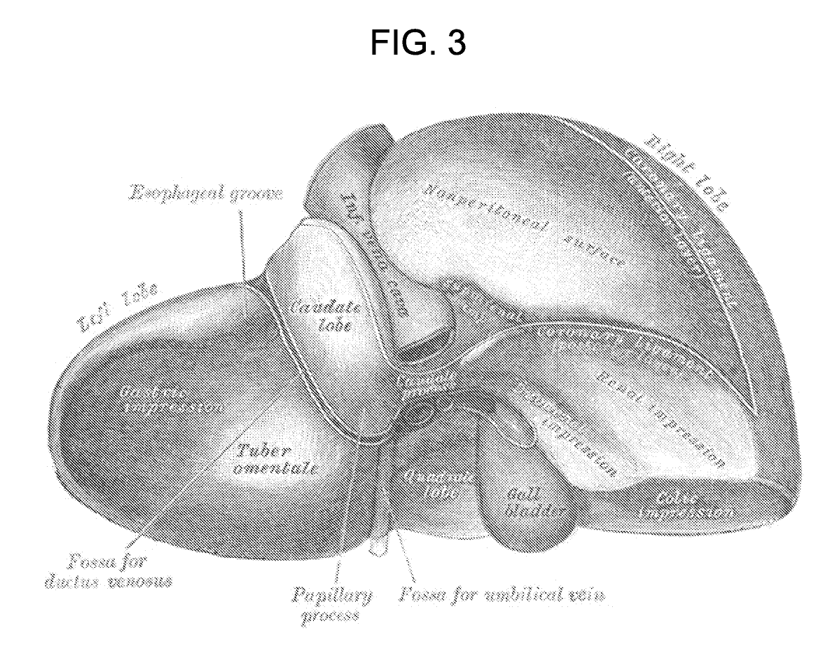

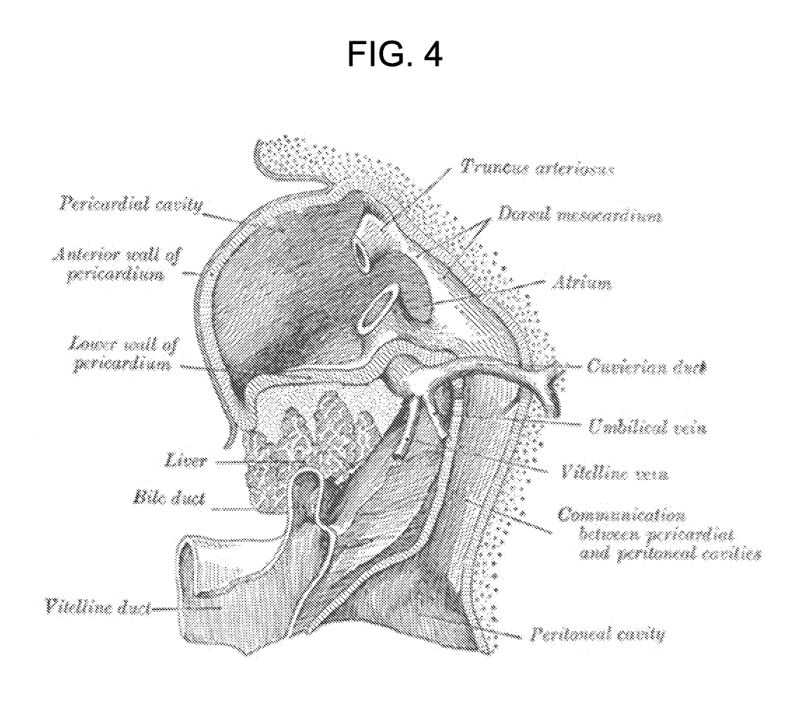

With reference to FIGS. 2-4, the scope of the invention further encompasses a method of treating a patient's biliary duct pain associated with hypertension of bile therein. The method includes applying an electrical stimulation signal to at least one smooth muscle of a patient's sphincter of Oddi whereby relaxation of said muscle is affected and reduced bile pressure in the patient's biliary duct is affected. This method may be applied preferably when the least one smooth muscle is located within a group of muscles, or the entirety of the muscle group, surrounding the distal end of the common bile duct and that moderates flow of bile and pancreatic fluids from the common bile duct into the digestive tract. Alternatively, this at least one smooth muscle may be one of, or the entirety of the group of muscles surrounding a portion of the bile duct that is proximal to the merging of the pancreatic duct with the distal portion of the bile duct. It should be understood that the appropriate group of muscles to be stimulated for a given patient will be determined by the diagnostic determination as to which of the sphincter components are pathological, or more specifically, in which portion of the biliary duct the pressure is being raised because of a failure of the bile to flow.

The scope of the present invention extends to treating a patient's biliary duct pain associated with hypertension of bile therein by applying an electrical stimulation signal to at least one nerve fiber, such that relaxation of at least one smooth muscle of a patient's sphincter of Oddi is affected and reduced bile pressure in the patient's biliary duct is affected. This may be achieved by applying said stimulatory signal to nerves emanating from a patient's sympathetic nerve chain. Alternatively, this may be achieved by applying the stimulation to nerve fibers emanating from the patient's tenth cranial nerve. It is preferable, however, that the stimulation be applied to the nerve plexus of fibers emanating from both the sympathetic nerve chain and the tenth cranial nerve, and this is most preferably the hepatic plexus.

The target muscles that are targeted in the above embodiment of the present invention may include at least one smooth muscle is located within a group of muscles surrounding the distal end of the common bile duct or among the muscles surrounding a portion of the bile duct that is proximal to the merging of the pancreatic duct with the distal portion of the bile duct. Again, the appropriate muscle or muscles to stimulate and/or affect by stimulation of the nerves that control its (their) function is determined by diagnostic testing to determine where the pathological tension and/or spasms are located.

In a related fashion, the present invention further encompassed a method of treating a patient's pancreatic pain associated with hypertension of pancreatic fluids comprising applying an electrical stimulation signal to at least one smooth muscle of a patient's sphincter of Oddi whereby relaxation of said muscle is affected and reduced pancreatic fluid pressure is affected. This muscle or muscles is typically found within the complex of muscles surrounding the distal end of the patient's duct through which pancreatic fluids flow prior to merging with the patient's bile duct to form the patient's common bile duct.

This treatment of pancreatic pain can be achieved also by relaxing the muscles described above, by applying an electrical stimulation signal to at least one nerve fiber, such that relaxation of at least one smooth muscle of a patient's sphincter of Oddi is affected, and reduced pressure of pancreatic fluids is affected. As above, this (these) nerve(s) can be one or more that from a patient's sympathetic nerve chain, or fibers of the tenth cranial nerve (that vagus nerve). Preferably, however, this stimulation would be applied to the hepatic plexus.

In this case, the smooth muscle that is the target is that which surrounds the distal end of the duct that moderates flow of pancreatic fluids from the pancreas into the common bile duct.

In a very appealing aspect, the present invention includes a method of reducing a patient's blood cholesterol levels. This method comprises applying an electrical stimulation signal to at least one smooth muscle of a patient's sphincter of Oddi whereby a tightening of said muscle is affected and reduced bile flow from the patient's common biliary duct into the patient's digestive tract is affected. This treatment can be applied to either the muscles surrounding the distal end of the common bile duct and that moderates flow of bile and pancreatic fluids from the common bile duct into the digestive tract, or to the muscles surrounding a portion of the bile duct that is proximal to the merging of the pancreatic duct with the distal portion of the bile duct. It is preferable that the muscles affected be the latter, as this will limit the extent to which reflux of bile may pass up into the duct connecting the pancreas to the common bile duct, however, should the control of this proximal duct not be clinically easy to manage, the distal sphincter muscles surrounding the terminus of the common bile duct, at the digestive tract, is acceptable as well.

This same effect, i.e., the reduction in blood cholesterol may be achieved within the scope of this invention by applying an electrical stimulation signal to at least one nerve fiber, such that tightening of at least one smooth muscle of a patient's sphincter of Oddi is affected and increased bile pressure in the patient's biliary duct is affected. This increased bile pressure translates into a higher local concentration of cholesterol in the vicinity of the cells that produce cholesterol. The presence of greater quantities of cholesterol, effectively trapped within the liver, serves as a feedback regulation signal to coenzyme HOA-C in the biosynthetic pathway for the production of cholesterol, serving to reduce the rate at which cholesterol is produced. This phenomenon is the result of cholesterol competitively binding to this coenzyme, thus reducing the number of new cholesterol molecules being synthesized. It shall be understood, however, that such a tightening of the sphincter of Oddi, or a portion thereof, should preferably be done at night only, during which time the body typically needs little bile for digestion. As this is not always the case, the application of this tightening should be limited to patient regulated control. As the patient is apt to forget the state of the stimulation, unless otherwise appraised of its activity by sensing the stimulation, the signal should have an automatic shut off that terminates the signal after a defined period of time.

The nerves that would be stimulated in this application are the same as with the other applications of the present invention related to the sphincter of Oddi, including at least one nerve emanating from a patient's sympathetic nerve chain and/or one or more nerves emanating from the patient's tenth cranial nerve. Again, this is more preferably applied through a nerve plexus of fibers emanating from both the sympathetic nerve chain and the tenth cranial nerve, which is often the hepatic plexus. Similarly, the smooth muscle affected should be at least one located within a group of muscles surrounding the distal end of the common bile duct and that moderates flow of bile and pancreatic fluids from the common bile duct into the digestive tract. Should reflux of bile into the pancreas or its duct be an issue, however, the muscles surrounding a portion of the bile duct that is proximal to the merging of the pancreatic duct with the distal portion of the bile duct may be targeted for tightening instead.

The free flow of bile into the gut when no food matter is present is a powerful stimulant of sensations of hunger. This is more dramatically exhibited in patients who have experienced a cholecystectomy, and/or a post cholecystectomy sphincterotomy. In these patients, hunger pains can reach significantly discomforting levels, waking them up in the middle of the night and all but requiring them to eat something in order to affect a subsidence of the pain. This additional food intake, followed by a return to sleep, can easily lead to obesity.

In fact, it has been proposed that patients who are obese, and who complain of a near constant hunger that drives them to eat, may suffer from low tonicity in the sphincter of Oddi that results in a constant flow of bile into the digestive tract, which has the effect of amplifying and accelerating the return of hunger pains after ingesting a meal. A method of reducing a patient's feelings of hunger and thereby affect weight loss that is consistent with the present invention, therefore, comprises applying an electrical stimulation signal to at least one smooth muscle of a patient's sphincter of Oddi whereby a tightening of said muscle is affected and reduced bile flow from the patient's common biliary duct into the patient's digestive tract is affected. This stimulation may be applied to the muscles surrounding the distal end of the common bile duct, or muscles surrounding a portion of the bile duct that is proximal to the merging of the pancreatic duct with the distal portion of the bile duct.

Similarly, this same effect may be generated by applying an electrical stimulation signal to nerve fibers, such that relaxation of at least one smooth muscle of a patient's sphincter of Oddi is affected and reduced bile pressure in the patient's biliary duct is affected. Again, these muscles can be the muscles surrounding the distal end of the common bile duct, or muscles surrounding a portion of the bile duct that is proximal to the merging of the pancreatic duct with the distal portion of the bile duct. This control is obtained by stimulating nerves emanating from a patient's sympathetic nerve chain and/or nerve fibers emanating from the vagus nerve. As the hepatic plexus is the node at which fibers from both the sympathetic nerve chain and the vagus nerve combine, this is an ideal location to stimulate these nerves.

It shall be understood that the stimulation of muscle tissue to contract (or in the case of a sphincter, to tighten) requires a different form of applied electrical signal than those typically used to relax muscle tissue. By way of example, U.S. Pat. No. 6,928,320 to King describes the various frequency ranges that have been found to be effective for relaxing and activating various tissues. The specification of U.S. Pat. No. 6,928,320 and the references cited therein are, therefore, incorporated by reference as examples of the various signal types that may be utilized to affect the therapeutic benefits encompassed by the present invention.

In all cases, however, the implanting surgeon should vary the signal generated by the stimulation driver unit and specific location of the lead until the desired outcome is achieved, and should monitor the long-term maintenance of this effect to ensure that adaptive mechanisms in the patient's body do not nullify the intended effects.

In one or more embodiments of the present invention, a treatment system may employ electrical signals to: (i) control functions like contracting and relaxing of one or more sphincters and/or structures of the gall bladder, pancreas, liver, bile duct, and/or Sphincter of Oddi system, or (ii) to release chemicals/hormones that influence sphincters. Electrical signals may be applied directly to the sphincters, surrounding tissue, nerve(s), plexus(es). Chemicals and/or hormones can be stimulated from the body or released from reservoirs that are part of the treatment system.

Command(s) to the digestive system can be based on: (i) patient input (e.g., through wireless telemetry or magnet/reed switch(es)) resulting from pain sensations or meal/bed time habits, etc.; (ii) responses to sensor data such as pressure in the patient's gall bladder or duct(s), nerve signals, stomach muscle signals, concentration of enzymes and/or hormones; (iii) physician pre-programmed schedules; and/or (iv) a default software program in the stimulator.

A valve and/or stent can be used to augment and/or replace damaged or diseased sphincters, ducts, etc. The valve opens and closes with an electrical signal based on the commands described above. The stent may be flexible so that sphincter contraction would still close the opening, or the stent material itself may respond to electrical signals to change shape. The stent may also be combined with a sensor to detect chemicals or pressure/flow information. The treatment system may have a stent/valve maintenance feature to periodically clean and flush debris using the bodies own fluids or a solution stored in the treatment system.

The electrical signals described above may be produced by an implanted generator or external stimulation device. The implanted generator may be powered and/or recharged from outside the body or may have its own power source.

The signals to the digestive system may be applied with leads and electrodes, or the electrodes could be part of a leadless generator(s) attached to parts of the digestive system. An external stimulation device may use magnetic induction coil or coils, or pads attached to the skin. Sensor data may be sent to the implanted generator via wires or wireless communication. Sensor data to an external device is sent by wireless telemetry.

The implanted generator system may have an external device for communication of settings to the generator and/or information from the generator to the external device. The external communication device and/or generator/stimulation device may store sensor data and/or stimulation signals and timing information. These devices may have a computer interface to download data to the computer for analysis and trending. Such data could also be used to modify the generator/stimulator programming to improve treatment.

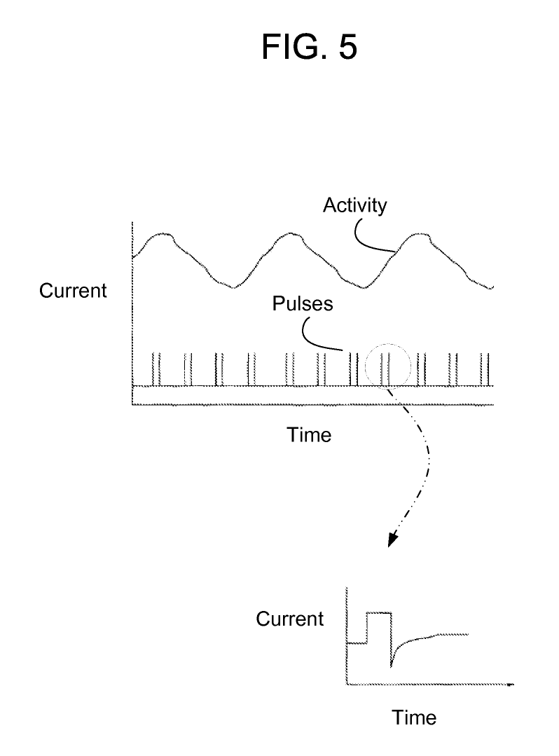

With reference to FIG. 5, the electrical voltage/current profile of the modulation signal to the electrodes (and thus the nerves/muscles) may be achieved using a pulse generator. In a preferred embodiment, the modulation unit includes a power source, a processor, a clock, a memory, etc. to produce a pulse train to the electrodes. The parameters of the modulation signal are preferably programmable, such as the frequency, amplitude, duty cycle, pulse width, pulse shape, etc. The modulation unit may be surgically implanted, such as in a subcutaneous pocket of the abdomen or positioned outside the patient. By way of example, the modulation unit may be purchased commercially, such as the Itrel 3 Model 7425 available from Medtronic, Inc. The modulation unit is preferably programmed with a physician programmer, such as a Model 7432 also available from Medtronic, Inc.

The modulation signal may have a frequency selected to influence the therapeutic result, such as from about 0.2 pulses per minute to about 18,000 pulses per minute, depending on the application. The modulation signal may have a pulse width selected to influence the therapeutic result, such as from about 0.01 ms to 500.0 ms. The modulation signal may have a peak current amplitude selected to influence the therapeutic result, such as from about 0.01 mA to 100.0 mA.

In addition, or as an alternative to the devices to implement the modulation unit for producing the electrical voltage/current profile of the modulation signal to the electrodes, the device disclosed in U.S. Patent Publication No.: 2005/0216062 (the entire disclosure of which is incorporated herein by reference), may be employed. U.S. Patent Publication No.: 2005/0216062 discloses a multi-functional electrical stimulation (ES) system adapted to yield output signals for effecting faradic, electromagnetic or other forms of electrical stimulation for a broad spectrum of different biological and biomedical applications. The system includes an ES signal stage having a selector coupled to a plurality of different signal generators, each producing a signal having a distinct shape such as a sine, a square or a saw-tooth wave, or simple or complex pulse, the parameters of which are adjustable in regard to amplitude, duration, repetition rate and other variables. The signal from the selected generator in the ES stage is fed to at least one output stage where it is processed to produce a high or low voltage or current output of a desired polarity whereby the output stage is capable of yielding an electrical stimulation signal appropriate for its intended application. Also included in the system is a measuring stage which measures and displays the electrical stimulation signal operating on the substance being treated as well as the outputs of various sensors which sense conditions prevailing in this substance whereby the user of the system can manually adjust it or have it automatically adjusted by feedback to provide an electrical stimulation signal of whatever type he wishes and the user can then observe the effect of this signal on a substance being treated.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of treating asthmatic spasms, comprising applying an electrical stimulation signal to at least one smooth muscle disposed in the vicinity of bronchial tissues of a patient, whereby relaxation of said at least one smooth muscle is affected to promote proper functioning of the bronchial tissues.

2. A method of treating asthmatic spasms of bronchial tissue, comprising applying an electrical stimulation signal to at least one nerve fiber such that relaxation of at least one smooth muscle disposed in the vicinity of a patient's bronchial tissues is affected to promote proper functioning of the bronchial tissue.

3. The method set forth in claim 2, wherein the at least one nerve fiber comprises at least one nerve emanating from the patient's sympathetic nerve chain.

4. The method set forth in claim 2, wherein the at least one nerve comprises at least one nerve fibers emanating from the patient's tenth cranial nerve.

5. The method set forth in claim 2 wherein the at least one nerve comprises a nerve plexus of fibers emanating from both a sympathetic nerve chain and a tenth cranial nerve of the patient.

6. The method set forth in claim 5 wherein the nerve plexus comprises an anterior pulmonary plexus.

7. The method set forth in claim 5 wherein the nerve plexus comprises a posterior pulmonary plexus.

8. A method of treating a patient's biliary duct pain, associated with hypertension of bile therein, comprising applying an electrical stimulation signal to at least one smooth muscle of the patient's sphincter of Oddi, whereby relaxation of said muscle is affected and reduced bile pressure in the patient's biliary duct is affected.

9. The method as set forth in claim 8, wherein the at least one smooth muscle is located within a group of muscles surrounding a distal end of a common bile duct and that moderates flow of bile and pancreatic fluids from the common bile duct into a digestive tract of the patient.

10. The method as set forth in claim 8, wherein the at least one smooth muscle is located within a group of muscles surrounding a portion of the biliary duct that is proximal to a merging of a pancreatic duct with a distal portion of the biliary duct.

11. A method of treating a patient's biliary duct pain, associated with hypertension of bile therein, comprising applying an electrical stimulation signal to at least one nerve fiber, such that relaxation of at least one smooth muscle of a patient's sphincter of Oddi is affected and reduced bile pressure in the patient's biliary duct is affected.

12. The method set forth in claim 11, wherein the at least one nerve fiber comprises at least one nerve emanating from the patient's sympathetic nerve chain.

13. The method set forth in claim 11, wherein the at least one nerve comprises at least one nerve fibers emanating from the patient's tenth cranial nerve.

14. The method set forth in claim 11, wherein the at least one nerve comprises a nerve plexus of fibers emanating from both a sympathetic nerve chain and a tenth cranial nerve of the patient.

15. The method as set forth in claim 14, wherein the at least one nerve plexus comprises the patient's hepatic plexus.

16. The method as set forth in claim 11, wherein the at least one smooth muscle is located within a group of muscles surrounding a distal end of a common bile duct and that moderates flow of bile and pancreatic fluids from the common bile duct into the patient's digestive tract.

17. The method as set forth in claim 16, wherein the at least one nerve plexus comprises the patient's hepatic plexus.

18. The method as set forth in claim 11, wherein the at least one smooth muscle is located within a group of muscles surrounding a portion of the biliary duct that is proximal to a merging of the patient's pancreatic duct with a distal portion of the biliary duct.

19. The method as set forth in claim 18, wherein the at least one nerve plexus comprises the patient's hepatic plexus.

20. A method of treating a patient's pancreatic pain, associated with hypertension of pancreatic fluids, comprising applying an electrical stimulation signal to at least one smooth muscle of the patient's sphincter of Oddi, whereby relaxation of said muscle is affected and reduced pancreatic fluid pressure is affected.

21. The method as set forth in claim 20, wherein the at least one smooth muscle is located within a group of muscles surrounding a distal end of the patient's duct through which pancreatic fluids flow prior to merging with the patient's bile duct to form the patient's common bile duct.

22. A method of treating a patient's pancreatic pain caused by hypertension of pancreatic fluids, comprising applying an electrical stimulation signal to at least one nerve fiber, such that relaxation of at least one smooth muscle of a patient's sphincter of Oddi is affected, and reduced pressure of pancreatic fluids is affected.

23. The method set forth in claim 22, wherein the at least one nerve fiber comprises at least one nerve emanating from the patient's sympathetic nerve chain.

24. The method set forth in claim 22, wherein the at least one nerve comprises at least one nerve fibers emanating from the patient's tenth cranial nerve.

25. The method set forth in claim 22 wherein the at least one nerve comprises a nerve plexus of fibers emanating from both the patient's sympathetic nerve chain and the patent's tenth cranial nerve.

26. The method as set forth in claim 25, wherein the at least one nerve plexus comprises the patient's hepatic plexus.

27. The method as set forth in claim 22, wherein the at least one smooth muscle is located within a group of muscles surrounding a distal end of a duct that moderates flow of pancreatic fluids from the patient's pancreas into the patient's common bile duct.

28. The method as set forth in claim 27, wherein the at least one nerve plexus comprises the patient's hepatic plexus.

29. A method of reducing a patient's blood cholesterol levels, comprising applying an electrical stimulation signal to at least one smooth muscle of the patient's sphincter of Oddi, whereby a tightening of said muscle is affected and reduced bile flow from the patient's common biliary duct into the patient's digestive tract is affected.

30. The method as set forth in claim 29, wherein the at least one smooth muscle is located within a group of muscles surrounding a distal end of the common biliary duct and that moderates flow of bile and pancreatic fluids from the common biliary duct into the patient's digestive tract.

31. The method as set forth in claim 29, wherein the at least one smooth muscle is located within the group of muscles surrounding a portion of the biliary duct that is proximal to a merging of the patient's pancreatic duct with a distal portion of the biliary duct.

32. A method of reducing a patient's blood cholesterol levels, comprising applying an electrical stimulation signal to at least one nerve fiber, such that tightening of at least one smooth muscle of a patient's sphincter of Oddi is affected and an increase in cholesterol concentration within the patient's liver is affected.

33. The method set forth in claim 32, wherein the at least one nerve fiber comprises at least one nerve emanating from the patient's sympathetic nerve chain.

34. The method set forth in claim 32, wherein the at least one nerve comprises at least one nerve fiber emanating from the patient's tenth cranial nerve.

35. The method set forth in claim 32 wherein the at least one nerve comprises a nerve plexus of fibers emanating from both the patient's sympathetic nerve chain and the patient's tenth cranial nerve.

36. The method as set forth in claim 35, wherein the at least one nerve plexus comprises the patient's hepatic plexus.

37. The method as set forth in claim 32, wherein the at least one smooth muscle is located within a group of muscles surrounding a distal end of the common biliary duct and that moderates flow of bile and pancreatic fluids from the common biliary duct into the digestive tract.

38. The method as set forth in claim 37, wherein the at least one nerve plexus comprises the patient's hepatic plexus.

39. The method as set forth in claim 32, wherein the at least one smooth muscle is located within a group of muscles surrounding a portion of the biliary duct that is proximal to a merging of the patient's pancreatic duct with a distal portion of the biliary duct.

40. The method as set forth in claim 39, wherein the at least one nerve plexus comprises the patient's hepatic plexus.

41. A method of reducing a patient's feelings of hunger and thereby affect weight loss, comprising applying an electrical stimulation signal to at least one smooth muscle of a patient's sphincter of Oddi, whereby a tightening of said muscle is affected and reduced bile flow from the patient's common biliary duct into the patient's digestive tract is affected.

42. The method as set forth in claim 41, wherein the at least one smooth muscle is located within a group of muscles surrounding a distal end of the common biliary duct and that moderates flow of bile and pancreatic fluids from the common biliary duct into the digestive tract.

43. The method as set forth in claim 41, wherein the at least one smooth muscle is located within a group of muscles surrounding a portion of the biliary duct that is proximal to a merging of the patient's pancreatic duct with a distal portion of the patient's biliary duct.

44. A method of reducing a patient's feelings of hunger and thereby affect weight loss, comprising applying an electrical stimulation signal to at least one nerve fiber, such that relaxation of at least one smooth muscle of the patient's sphincter of Oddi is affected and reduced bile pressure in the patient's biliary duct is affected.

45. The method set forth in claim 44, wherein the at least one nerve fiber comprises at least one nerve emanating from the patient's sympathetic nerve chain.

46. The method set forth in claim 44, wherein the at least one nerve comprises at least one nerve fiber emanating from the patient's tenth cranial nerve.

47. The method set forth in claim 44 wherein the at least one nerve comprises a nerve plexus of fibers emanating from both the patient's sympathetic nerve chain and the patient's tenth cranial nerve.

48. The method as set forth in claim 47, wherein the at least one nerve plexus comprises the patient's hepatic plexus.

49. The method as set forth in claim 44, wherein the at least one smooth muscle is located within a group of muscles surrounding a distal end of the patient's common biliary duct and that moderates flow of bile and pancreatic fluids from the common bile duct into the digestive tract.

50. The method as set forth in claim 49, wherein the at least one nerve plexus comprises the patient's hepatic plexus.

51. The method as set forth in claim 44, wherein the at least one smooth muscle is located within a group of muscles surrounding a portion of the biliary duct that is proximal to a merging of the patient's pancreatic duct with a distal portion of the biliary duct.

52. The method as set forth in claim 51, wherein the at least one nerve plexus comprises the patient's hepatic plexus.
